(19) United States
(12) Patent Application Publication
(10) Pub. No.: US 2008/0075792 A1
(43) Pub. Date: Mar. 27, 2008

(54) Method for inhibiting the activity of DPP-IV and lowering blood glucose level

(75) Inventor: Jae Soo Kim, Koyang (KR)

Correspondence Address: CLARK & ELBING LLP, 101 FEDERAL STREET, BOSTON, MA, 02110, US

(73) Assignee: Naturalendo Tech Co., Ltd., Seoul (KR)

(21) Appl. No.: 11/524,713

(22) Filed: Sep. 21, 2006

(51) Int. Cl. A61K 36/899; A61K 36/28; A61K 36/67

(52) U.S. Cl. 424/725; 424/734; 424/750; 424/764

(57) ABSTRACT

The present invention relates to a method for inhibiting the activity of dipeptidyl peptidase-IV (DPP-IV) and decreasing blood glucose levels.

### BACKGROUND OF THE INVENTION

1. Field of the Invention

The present invention relates to a method for inhibiting the activity of dipeptidyl peptidase-IV (DPP-IV) and decreasing a blood glucose level.

2. Background of the Related Art

Incretin hormone is a hormone to facilitate secretion of insulin in response to nutrient digestion of the small intestines and is classified into two types of GIP (Glucose-dependent insulinotropic polypeptide) and GLP-1 (Glucagon like peptide-1).

GLP-1 is composed of total 30 amino acids and is involved in various homeostasis such as secretion and biological synthesis of insulin which is secreted in response to nutrient digestion to participate in glucose metabolism, inhibition of glucagon secretion, inhibition of food intake (inhibition of secretion and movement of the digestive organs, particularly inhibition of movement of the stomach and intestines).

Both GIP and GLP-1 are indispensable hormones to maintain normal glucose metabolism. However, patients with type 2 diabetes maintain normal or nearly normal GIP level in blood but show a reduced GLP-1 secretion level. GLP-1 is one of the strongest factors to increase insulin secretion and shows about 10 pmol/L of EC₅₀ (half-maximal effective concentration).

It was reported in 1992 for the first time that the use of GLP-1 is effective in treatment of diabetes. According to the report, while GLP-1 is dropped in the artificial pancreas, the blood glucose level is maintained at a normal level without need of insulin. Patients with type 2 diabetes who had experienced secondary failure on Oral Hypoglycemic agent were subject to GLP-1 dropping for 4 hours and showed complete normalization in the blood glucose level. Thus, GLP-1 was expected to be a treating agent of diabetes. Though the patients showed an average blood glucose level on an empty stomach of 13 mmol/L and a high HbAlc level, GLP-1 was effective.

From the continuous studies afterwards, it was found that administration of GLP-1 to the patients with type 2 diabetes was effective in decreasing a blood glucose level without regard to severeness of diabetes, duration of illness, types of previous treatment, complication company. Also, when patients with type 2 diabetes were intravenously administered with GLP-1 continuously for 1 week, they showed nearly normal blood glucose level even under condition of regular diet.

GLP-1 is synthesized in the endocrine cells of the viscera and has two main forms of “7-36 amide” and “7-37 amide”. The synthesized GLP-1 is digested by an enzyme called dipeptidyl peptidase IV (DPP-IV) and thereby, has 2 amino acids in the N-terminal excised and loses its activity.

DPP-IV excise proline and alanine from the N-terminal of GLP-1. However, simple change of their positions shows resistance against DPP-IV and elongated effective duration compared to living GLP-1.

The DPP-IV inhibiting agent has been developed, based on the fact that inhibition of DPP-IV activity in patients with type 2 diabetes increases GLP-1 level, thereby reducing the blood glucose level (Improved Glucose Tolerance via Enhanced Glucose-Dependent Insulin Secretion in Dipeptidyl Peptidase IV-Deficient Fischer Rats. Biochem Biophys Res Commun. 8; 284(2):501-6(2001); and Marguet D, et al., Enhanced insulin secretion and improved glucose tolerance in mice lacking CD26. Proc Natl Acad Sci USA 97(12):6874-6879(2000)). Thus, if there is a substance capable of selectively inhibiting DPP-IV, exogenous or endogenous decomposition of GLP-1 can be prevented to promote insulin secretion and thereby reduction of the blood glucose level.

One of the representative DPP-IV target compounds is LAF237. Recently, Novartis AG has conducted phase 2 clinical trial on patients with type 2 diabetes for 12 weeks. As a result, single administration or combined administration with Metformin showed excellent lowering effect of the blood glucose level and thus, phase 3 clinical trial is in progress. Meanwhile combined administration of Metformin with LAF237 or placebo showed HbAlc difference of 1.1±0.2% (p<0.0001) after 52 weeks.

However, there has not been conducted a research for natural materials to inhibit DPP-IV activity.

Throughout this application, various patents and publications are referenced and citations are provided in parentheses. The disclosure of these patents and publications in their entities are hereby incorporated by references into this application in order to more fully describe this invention and the state of the art to which this invention pertains.

### SUMMARY OF THE INVENTION

Accordingly, the present inventors have made every effort to develop a substance capable of inhibiting dipeptidyl peptidase-IV (DPP-IV) activity, thereby lowering abnormally high blood glucose level and consequently, have found that an extract from Piper longum and an extract from Marrubium vulgare, which are used as oriental herbal medicines, have the above-described effects. On the basis of the founding, the present invention has been completed.

Therefore, it is an object of the present invention to provide a composition for inhibiting dipeptidyl peptidase-IV (DPP-IV) activity.

It is another object of the present invention to provide a food composition for decreasing a blood glucose level.

It is still another object of the present invention to provide a pharmaceutical composition for decreasing a blood glucose level.

It is yet another object of the present invention to provide a method for inhibiting dipeptidyl peptidase-IV (DPP-IV) activity.

It is a further object of the present invention to provide a method for decreasing a blood glucose level.

Other objects and advantages of the present invention will become apparent from examples to follow, appended claims and drawings.

To accomplish the above objects, in an aspect of the present invention, there is provided a composition for inhibiting dipeptidyl peptidase-IV (DPP-IV) activity comprising at least one selected from the group consisting of an extract from Piper longum, an extract from Marrubium vulgare and an extract from Oryzasativa var. glutinosa, as an active ingredient.

In another aspect of the present invention, there is provided a food composition for blood glucose level comprising at least one selected from the group consisting of an extract from Piper longum, an extract from Marrubium vulgare and an extract from Oryzasativa var. glutinosa, as an active ingredient.

In a further aspect of the present invention, there is provided a pharmaceutical composition for decreasing a blood glucose level comprising (a) an effective amount of at least one selected from the group consisting of an extract from Piper longum, an extract from Marrubium vulgare and an extract from Oryzasativa var. glutinosa; and (b) a pharmaceutically acceptable carrier.

In further aspect of this invention, there is provided a method for inhibiting the activity of dipeptidyl peptidase-IV (DDP-IV) in a subject, which comprises administering to the subject an effective amount of a composition comprising as an active ingredient an extract from Piper longum, an extract from Marrubium vulgare, an extract from Oryzasativa var. glutinosa or its combination.

In still further aspect of this invention, there is provided a method for decreasing a blood glucose level in a subject, which comprises administering to the subject an effective amount of a composition comprising as an active ingredient an extract from Piper longum, an extract from Marrubium vulgare, an extract from Oryzasativa var. glutinosa or its combination.

The present inventors have screened inhibiting effect of various plant extracts on dipeptidyl peptidase-IV (DPP-IV) activity and finally, have found that an extract from Piper longum, an extract from Oryzasativa var. glutinosa and an extract from Marrubium vulgare inhibit DPP-IV activity, thereby showing effect of lowering the blood glucose level.

Piper longum used as an active ingredient in the composition of this invention has been used in treatment of the dismals, the intestinal convulsion, the congestive chill and precordial pain (Indian Drugs, June, 384-388, 1984), and largely cultivated in India and imported to Korea to be used as a herb for oriental medicines. Piper longum has been studied considerably for its physiological activity and has been reported to have several medical effects such as anti-allergy effect (Indian J. Physiol. Pharmacol., 43:486-490, 1999), anti-inflammation effect (Indian J. Exp. Biol., 32: 633-636, 1994), liver protecting effect (Planta Med., 59: 413-417, 19 93) and the like.

Marrubium vulgare (Horehound) used as another active ingredient in the composition of this invention has been in treatment of wounds, lack of appetite and digestive disorder.

Oryzasativa var. glutinosa (Oryzae Radix) used as another active ingredient in the composition of this invention is dried root or dried root stem of Oryzasativa var. glutinosa), which is an annual plant belonging to Family Gramineae. This herb is commonly prescribed to chronic hepatitis, perspiration or in symptoms of slight fever and cold sweat in the afternoon caused by phthisis. In this specification, the terms “Oryzasativa var. glutinosa” is Oryzae Radix, not defined otherwise.

The extracts from Piper longum, an extract from Marrubium vulgare, or an extract from Oryzasativa var. glutinosa are obtained using various extraction solvents: (a) water, (b) absolute or water-bearing lower alcohol containing 1-4 carbon atoms (methanol, ethanol, propanol, butanol, etc.), (c) mixture of lower alcohol and water, (d) acetone, (e) ethyl acetate, (f) chloroform, (g) 1,3-butylene glycol and (h) butyl acetate. Most preferably, the extracts of this invention is obtained using ethanol or mixture of ethanol and water as extraction solvent. Furthermore, it is apparent to one skilled in the art that other conventional solvents may be employed for substantially identical extraction efficiency.

The extracts of this invention include those subject to additional purification by the well-known methods in the art as well as those obtained by extraction. For instance, it could be appreciated that active fractions obtained using a variety of additional purification methods such as an ultrafiltration with defined molecular weight cut-off value and various chromatography (designed for purification dependent upon size, charge, hydrophobicity and affinity) are included in the present extracts.

The extracts of this invention can be obtained in the form of powder by use of vacuum distillation, lyophilization or spray drying.

The effect of the extract from Piper longum, the extract from Marrubium vulgare and the extract from Oryzasativa var. glutinosa according to the present invention to inhibit dipeptidyl peptidase-IV (DPP-IV) activity is particularly accomplished by inhibiting binding of DPP-IV to a substrate of a normal living body. Thus, the extract from Piper longum, the extract from Marrubium vulgare and the extract from Oryzasativa var. glutinosa inhibits DPP-IV activity by working as a competitive inhibitor.

The blood glucose level lowering effect of the extract from Piper longum, the extract from Marrubium vulgare and the extract from Oryzasativa var. glutinosa means the action to reduce the abnormally increased blood glucose level.

In the pharmaceutical compositions of this invention, the pharmaceutically acceptable carrier may be conventional one for formulation, including carbohydrates (e.g., lactose, amylose, dextrose, sucrose, sorbitol, mannitol, starch, cellulose), gum acacia, calcium phosphate, alginate, gelatin, calcium silicate, microcrystalline cellulose, polyvinylpyrrolidone, water, salt solutions, alcohols, gum arabic, syrup, vegetable oils (e.g., corn oil, cotton-seed oil, peanut oil, olive oil, coconut oil), polyethylene glycols, methyl cellulose, methylhydroxy benzoate, propylhydroxy benzoate, talc, magnesium stearate and mineral oil, but not limited to. The pharmaceutical compositions of this invention, further may contain wetting agent, sweetening agent, emulsifier, buffer, suspending agent, preservatives, flavors, perfumes, lubricant, stabilizer, or mixtures of these substances.

The pharmaceutical composition of this invention may be administered orally or parenterally. For non-oral administration, intravenous injection, subcutaneous injection, intramuscular injection, nasal spray, sublingual spray may be employed. Preferred method is oral administration or sublingual spray and the most preferred is oral administration.

The correct dosage of the pharmaceutical compositions of this invention will be varied according to the particular formulation, the mode of application, age, body weight and sex of the patient, diet, time of administration, condition of the patient, drug combinations, reaction sensitivities and severity of the disease. It is understood that the ordinary skilled physician will readily be able to determine and prescribe a correct dosage of this pharmaceutical compositions. According to a preferred embodiment of this invention, a suitable dosage unit for human host is to administer once a day with the composition of 0.001-100 mg/kg.

According to the conventional techniques known to those skilled in the art, the pharmaceutical compositions of this invention can be formulated with pharmaceutical acceptable carrier and/or vehicle as described above, finally providing several forms including a unit dosage form. Non-limiting examples of the formulations include, but not limited to, a solution, a suspension or an emulsion, an extract, an elixir, a powder, a granule, a tablet, a capsule, emplastra, a liniment, a lotion and an ointment.

In the food composition of this invention, it can comprise typical ingredients incorporated into food products known to one skilled in the art. For example, for preparation of drinks, citric acid, liquid fructose, sucrose, glucose, acetic acid, malic acid, fruit juice, Eucommiae Cortex extract, Zizyphus jujuba extract and Glycyrrhiza, Liquorice extract may be further included in addition to an extract from Piper longum, an extract from Marrubium vulgare, and/or an extract from Oryzasativa var. glutinosa. A food of the present invention is very effective in lowering a blood glucose level.

The composition of this invention inhibits DPP-IV activity by working as a competitive inhibitor of DPP-IV and increases GLP-1 (Glucagon like peptide-1) activity in a living body, thereby promoting insulin secretion. Thus, the abnormally increased blood glucose level is effectively reduced and acts as a therapeutic agent for diabetes, particularly, type 2 diabetes. Also, since the composition of this invention uses, as an effective ingredient, plant extracts which has been used in oriental herbal medicines, it does not cause damage to human bodies.

Meanwhile, the composition of this invention can be administered in combination with any existing medicines which have been developed as treating agents for type 2 diabetes (for example, sulfonyl urea, Metformin) to show synergic blood glucose level lowering effect, treatment of diabetes, particularly type 2 diabetes.

### BRIEF DESCRIPTION OF THE DRAWINGS

The application file contains drawings executed in color. Copies of this patent or patent application with color drawing(s) will be provided by the Office upon request and payment of the necessary fee.

The above and other objects, features and advantages of the present invention will be apparent from the following detailed description of the preferred embodiments of the invention in conjunction with the accompanying drawings, in which:

FIG. 1 is a graph showing that the extract from Piper longum and the extract from Marrubium vulgare according to the present invention inhibits dipeptidyl peptidase-IV (DPP-IV) activity;

FIG. 2 is a graph analyzing DPP-IV activity in serum;

FIG. 3 to FIG. 5 shows a result of the experiment analyzing the inhibition of the extracts according to the present invention on the DPP-IV activity, in which M1, M2, M3, M4 and M5 are each an extract from Oryzasativa var. glutinosa with water, an extract from Oryzasativa var. glutinosa with ethanol, an extract from Piper longum with water, an extract Marrubium vulgare with water and an extract Piper longum with ethanol and the extract used in FIG. 4 has a concentration of 100 μg/ml;

FIG. 6 is a graph showing that the extract from Piper longum according to the present invention can decrease the blood glucose level;

FIG. 7 is a graph showing that the extract from Marrubium vulgare according to the present invention can decrease the blood glucose level; and

FIG. 8 is a graph showing that the extract from Oryzasativa var. glutinosa according to the present invention can decrease the blood glucose level.

### DETAILED DESCRIPTION OF THE PREFERRED EMBODIMENT

The following specific examples are intended to be illustrative of the invention and should not be construed as limiting the scope of the invention as defined by appended claims.

### Example 1

### Assay of Lowering Effect on Dipeptidyl Peptidase-IV Activity I

In the following Example 2, the effect of lowering the dipeptidyl peptidase-IV (DPP-IV) activity was assayed as follows.

Firstly, in order to form a thin layer of DPP-IV, the functional group was substituted with SPDP (intermediate for substitution of functional group, 3-(2-Pyridyldithio)propionic acid N-hydroxysuccinimide ester). 1 mM DPP-IV (Sigma, USA) and 1 mM SPDP (Sigma, USA) were mixed in a volumic rate of 1:1 and the mixture was filtered through gel. By the mixing, amine group of DPP-IV formed a cross-linked structure with a part of SPDP (2-pyridyldthio-propionate) with succinimide group removed. The cross-linked DPP-IV mixture was mixed with 1 mM of DTT (dithiothreitol) to reduce disulfide group into thiol group. A golden substrate was impregnated with the DPP-IV solution with functional groups substituted to prepare a thin layer having DPP-IV attached onto the surface.

The DPP-IV thin layer was impregnated with a solution containing a test sample to be assayed (1 mM) and GFP (green fluorescent protein, Clontech, USA; 1 mM) and reacted for 24 hours at room temperature. If the test sample contained NH, the DPP-IV recognized NH and DPP-IV(+) and GFP(−) each acted through charges. The DPP-IV which had reacted with the test sample containing NH lost its own charge due to ion transition between molecules and formed weak binding to GFP by charges, thereby difference in fluorescence intensity as compared to DPP-IV which had not reacted with the test sample. GFP emission wavelength was measured 510 nm which was its peculiar wavelength range.

### Example 2

### Screening of Inhibitor of Dipeptidyl Peptidase-IV Activity

Plant extracts capable of inhibiting dipeptidyl peptidase-IV (DPP-IV) activity were screened.

Firstly, Papaver somniferum, Chelidonium majus var. asiaticum, Oryzasativa var. glutinosa, Piper longum and Marrubium vulgare were selected for the screening.

Extracts from the above-described plants were obtained as follows: 40 g of Piper longum and Marrubium vulgare were measured using an electronic balance and pulverized with a grinder to form pieces or powder. The pulverized materials were divided into 20 g using an electronic balance. The divided 20 g of materials were extracted with a solvent mixture of water and ethanol. The extraction was performed by stirring the solution on a Hot plate stirrer for 2 hours. After completion of stirring, micro extraction was performed for 2 hours using supersonic waves. After completion of extraction, the solution was filtered using a 0.45 μm filter to remove impurities.

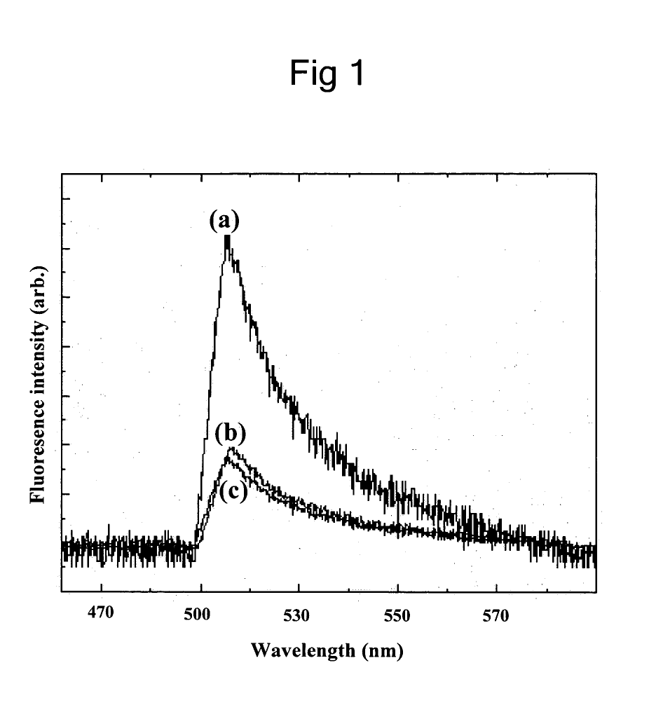

The obtained extracts were examined for the DPP-IV inhibiting effect as described in Example 1 and the result are shown in FIG. 1. As shown in FIG. 1, the pure DPP-IV (a) showed a high fluorescence intensity as compared to other two cases since there occurred no other variation and thereby, charge binding to GFP was formed. Meanwhile, in the Piper longum extract (b) and the Marrubium vulgare extract (c), (+) charge of DPP-IV was lost due to interaction with DPP-IV, whereby weak charge binding of DPP-IV to GFP was formed and the fluorescence was low.

Therefore, it was known that the Piper longum extract and the Marrubium vulgare extract acted as a competitive inhibitor of DPP-IV and inhibited DPP-IV activity.

### Example 3

### Assay of Inhibiting Effect on Dipeptidyl Peptidase-IV Activity II

Unlike the above Examples 1 and 2, an in-vitro assay of inhibiting effect on dipeptidyl peptidase-IV was performed as follows: 1.9 g of MgCl₂, 1.488 g of HEPES, 2.3 g of NaCl and 2.5 g of BSA (bovine serum albumin) were added to 250 ml of distilled water form a reaction solution containing 80 mM MgCl₂, 25 mM HEPES, 140 mM NaCl and 1% BSA. Rat serum was used as a source of dipeptidyl peptidase-IV and Ala-Pro-AFC (7-amido-4-trifluoromethylcoumarin, Sigma-Aldrich) was used as a substrate.

60 μl of rat serum was mixed with 60 μl of a test sample to be assayed and 30 μl of the reaction solution and reacted for 10 minutes at room temperature. Then, the reaction mixture was mixed with Ala-Pro-AFC (final concentration of 40 μM) and reacted for 30 minutes at room temperature. The reaction was quenched by adding 50 μl of 25% acetic acid and the fluorescence was measured at 380-460 nm.

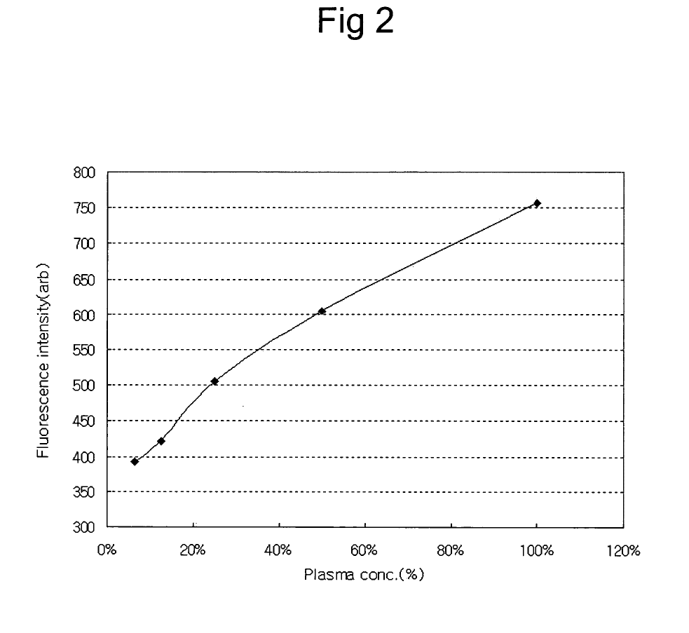

FIG. 2 is a graph analyzing DPP-IV activity in serum. As shown in FIG. 2, the DPP-IV activity was serum concentration-dependently increased.

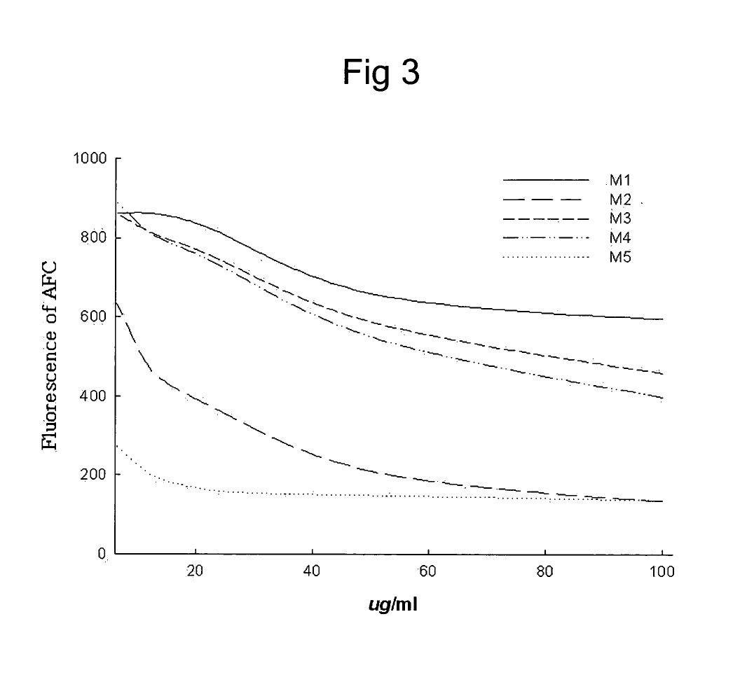

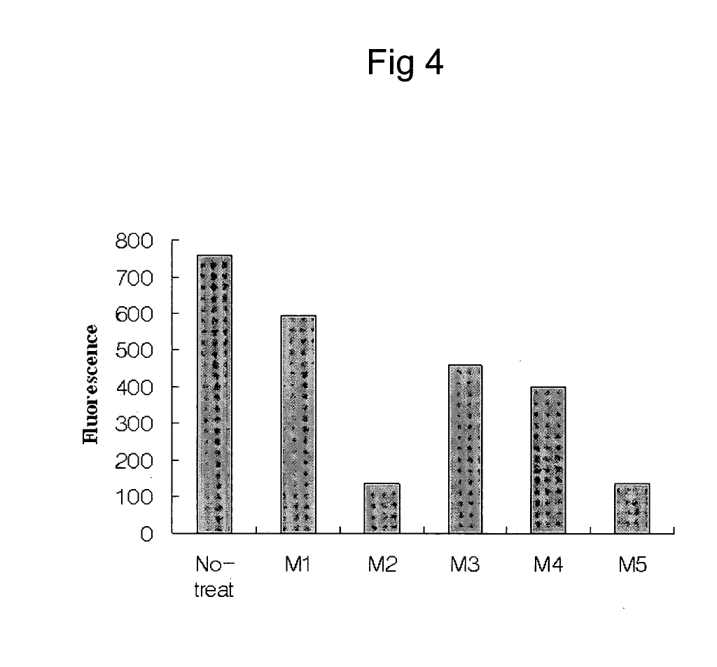

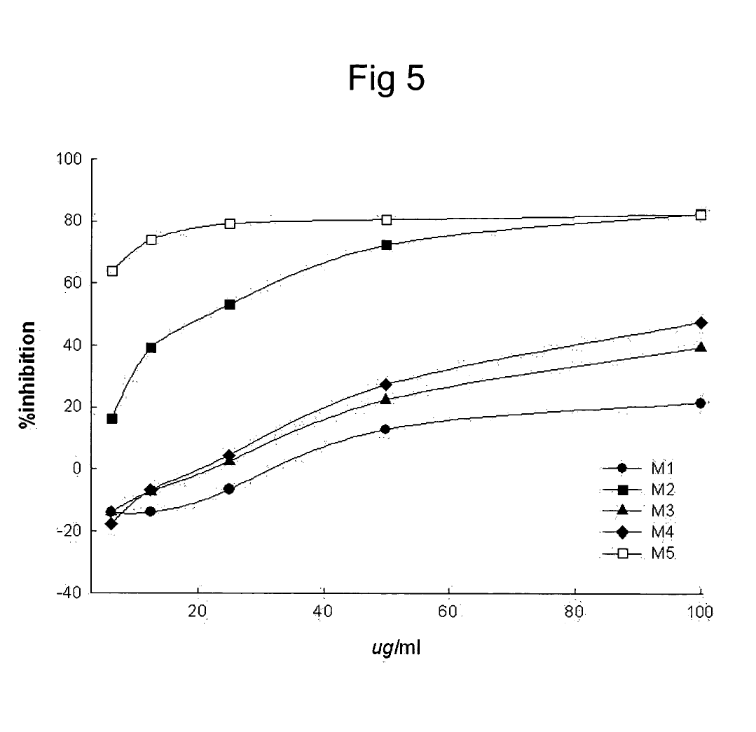

FIG. 3 to FIG. 5 shows a result of the experiment analyzing the inhibiting effect of the extracts according to the present invention on the DPP-IV activity. As shown in FIG. 3 to FIG. 5, the Oryzasativa var. glutinosa extract, the Piper longum extract and the Marrubium vulgare extract inhibited the DPP-IV activity concentration-dependently. Particularly, the Piper longum extract with ethanol showed the greatest inhibiting effect and the Oryzasativa var. glutinosa extract with ethanol also showed a high inhibiting effect.

In conclusion, in was noted that the Oryzasativa var. glutinosa extract, the Piper longum extract and the Marrubium vulgare extract according to the present invention could effectively inhibit the DPP-IV activity.

### Example 4

### Animal Test

Male Wistar rats (Charles river, Japan) were raised at 25° C. and a humidity of 30 to 70%, fasted for 24 hours and acclimated 1 hour before the experiment. The Piper longum extract, the Marrubium vulgare extract and the Oryzasativa var. glutinosa extract obtained in the above Example 2 were dried to form powder. The dried powder was mixed with water to form liquid for oral administration. At 4 hours prior to the oral administration of glucose (2 g/kg), each extract was administered in amounts of 10 mg/kg and 20 mg/kg. Blood was intravenously taken from the tale at −4, −0.5, 0, 0.5, 1 and 2 hours. The blood glucose level was assayed according to the standard glucose oxidase method.

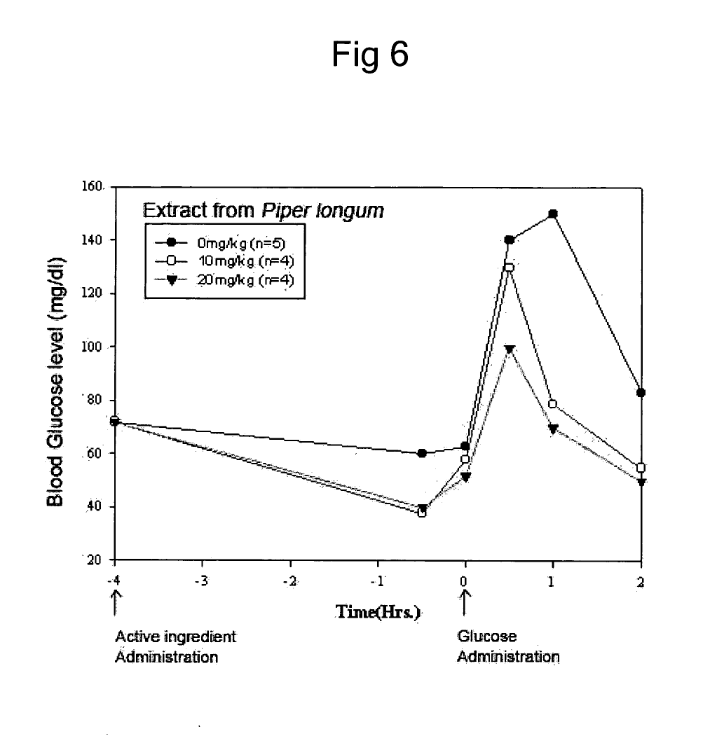

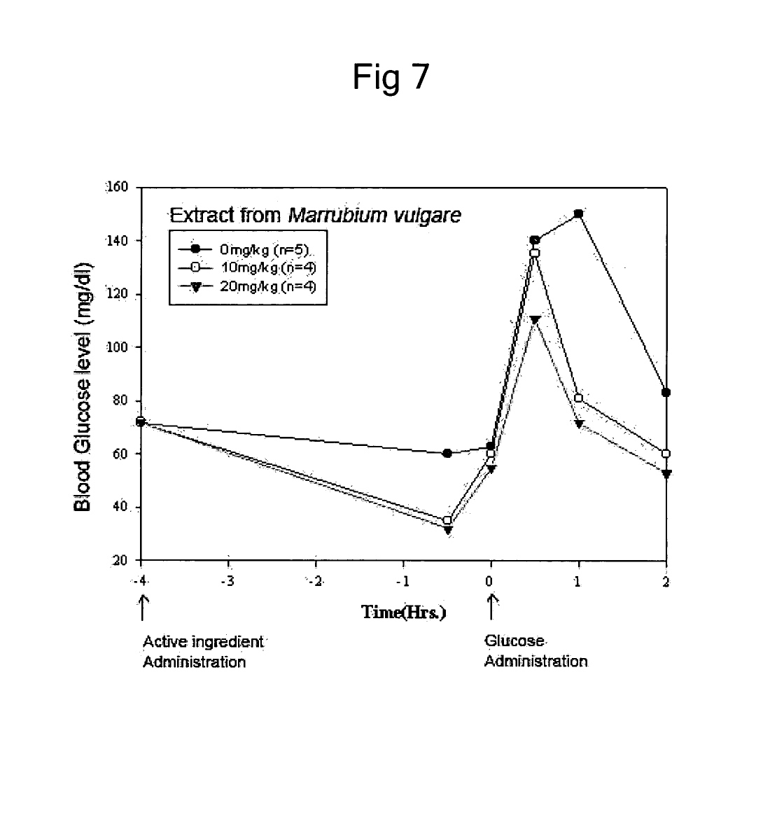

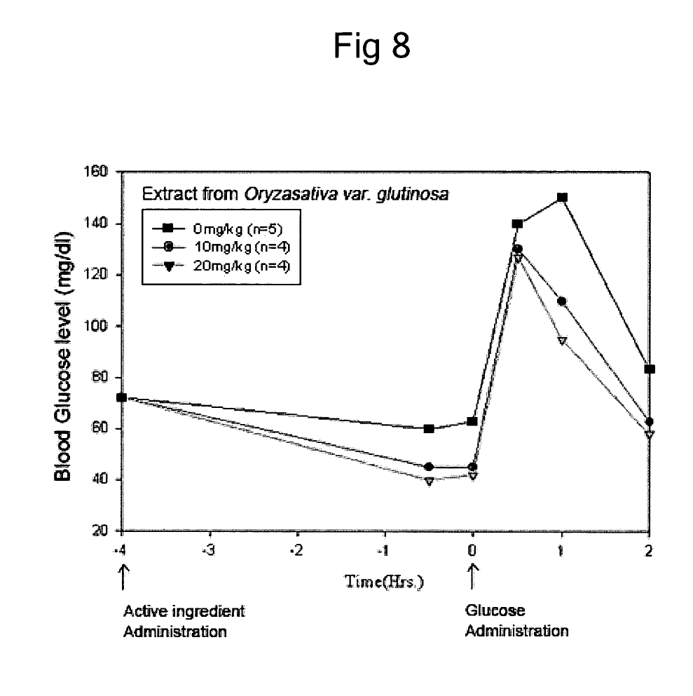

As confirmed in FIG. 6, FIG. 7 and FIG. 8, the rats which had not been administered with the extracts according to the present invention showed the maximum blood glucose level of 150 mg/dl at 1 hour after the glucose administration. However, the rats which had been administered with the Piper longum extract, the Marrubium vulgare extract and the Oryzasativa var. glutinosa extract in amounts of 10 mg and 20 mg showed reduced blood glucose levels of 60-80 mg/dl.

Therefore, it was noted that the Piper longum extract, the Marrubium vulgare extract and the Oryzasativa var. glutinosa extract according to the present invention could effectively reduce the increased blood glucose level.

In summarizing the results of the Examples 2-4, the Piper longum extract, the Marrubium vulgare extract and the Oryzasativa var. glutinosa extract according to the present invention inhibits the DPP-IV activity, thereby causing increase in GLP-1 (Glucagon like peptide-1) activity in living bodies and promotion of insulin secretion. Ultimately, the abnormally increased blood glucose level can be reduced.

What is claimed is:

1. A method for inhibiting the activity of dipeptidyl peptidase-IV (DDP-IV) in a subject, which comprises administering to the subject an effective amount of a composition comprising as an active ingredient an extract from Piper longum, an extract from Marrubium vulgare, an extract from Oryzasativa var. glutinosa or its combination.

2. A method for lowering a blood glucose level in a subject, which comprises administering to the subject an effective amount of a composition comprising as an active ingredient an extract from Piper longum, an extract from Marrubium vulgare, an extract from Oryzasativa var. glutinosa or its combination.
